(19) United States
(12) Patent Application Publication
(10) Pub. No.: US 2013/0236951 A1
(43) Pub. Date: Sep. 12, 2013

(54) Microalgae for Removal of Carbon Dioxide Generated from Biogas and Biogas Electric Generator

(71) Applicant: NATIONAL CHIAO TUNG UNIVERSITY, Hsinchu City (TW)

(72) Inventors: Ching-Ping TSENG, Hsinchu City (TW); Chih-Sheng LIN, Miaoli County (TW); Chiun-Hsun CHEN, Hsinchu City (TW)

(73) Assignee: NATIONAL CHIAO TUNG UNIVERSITY, Hsinchu City (TW)

(21) Appl. No.: 13/789,141

(22) Filed: Mar. 7, 2013

(30) Foreign Application Priority Data
Mar. 7, 2012 (TW) 101107655

(51) Int. Cl. C12M 1/107

(52) U.S. Cl. CPC C12M 21/04
USPC 435/266; 435/292.1

(57) ABSTRACT

The present invention relates to biogas and biogas electric generator and a biogas electricity generation method by use microalgae for removal of carbon dioxide generated from biogas and biogas electric generator. The electric generator integrates biogas production and purification, microalga culture, electricity generation, heat recycling and others into a unit volume, and, during microalgae culturing, uses carbon dioxide contained in biogas and that produced from electricity generation as a carbon source for photosynthesis to reduce the carbon dioxide contained in the biogas and the electricity generation exhaust gas, thereby attaining the goal of zero carbon emission.

This application claims the benefit of filing date of Taiwan Application Number 101107655, entitled “Biogas Electric Generator and Electricity Generation Method Using Microalgae Carbon Capture” filed Mar. 7, 2012 under 35 USC §119(e)(1).

### BACKGROUND OF THE INVENTION

1. Field of the Invention

The present invention relates to the use of microalgae cultures in removing carbon dioxide emitting from biogas and biogas electric generator.

2. Description of Related Art

Biogas is a cheap, environmentally-friendly renewable energy, appropriate for use in the generation of thermal energy, electric energy, and chemical substances or the application of automobile energy. Generally speaking, this type of biogas usually is composed of 50% to 70% methane, 20% to 30% carbon dioxide, 4% to 5% nitrogen, 0.2% to 0.5% hydrogen sulfide and other trace amount of gases, for which methane is one of the major greenhouse gases, when methane is released into the air, it is 20 times more effective than carbon dioxide in trapping heat in the atmosphere. In essence, it is a green energy of great economic potentials if it is collected and used effectively because discharge of greenhouse gases can be reduced, and application of renewable energy can be enhanced.

Biogas can come from agricultural waste or animal by-products and others produced by anaerobic fermentation. Based on the amount of available biogas concentration and economic benefits and other factors, the common knowledge about use of gas can be categorized into three types: (1) direct combustion: appropriate for family-friendly stores, lightning devices or boilers; (2) electric power generation: coupled with combustion by power generator to produce electric power; and (3) pipeline gas: produced from after purification, the quality of which is similar to natural gas, appropriate for civilian or industrial scale fuel. Nevertheless, even though combustion can work to reduce the greenhouse effect instigated by methane, the carbon dioxide produced therefrom will not be any less, and can hurt effort for zero carbon emission, and still lead to build up of greenhouse effect.

Accordingly it is desirable for biogas and biogas electric generator using microalgae carbon capture, to reduce carbon dioxide discharge in a process, and to further avoid acceleration of greenhouse effect during a power generation process.

### SUMMARY OF THE INVENTION

It is a major object of the present invention to provide biogas and biogas electric generator using carbon capture, which involves incorporating biogas generation, biogas purification, microalgae culture, electricity generation, heat recycling, and others into the confine of one single system, and using carbon dioxide included in biogas and that produced in electricity generation during the microalgae growing process, as the carbon source for photosynthesis, reducing the carbon dioxide included in biogas and waste gas from electricity generation, in order to reach the zero carbon emission objective.

In order to achieve the above object, an embodiment of the present invention is to provide biogas and biogas electric generator using microalgae carbon capture, comprising: a biogas production unit, which provides a first biogas, wherein, the first biogas comprises hydrogen sulfide, methane and carbon dioxide; a biogas purification unit, which receives the first biogas and removes the hydrogen sulfide of the first biogas, and discharges a second biogas; a microalgae culture unit, which houses a type of microalgae and receives the second biogas, wherein, the microalgae uses the carbon dioxide of the second biogas as carbon source to carry out photosynthesis before the microalgae culture unit discharges a third biogas; and an electric generation unit, which receives the third biogas for use as a fuel to generate electric energy, wherein, the electric energy powers the biogas electric generator.

When the gas produced by the generator includes carbon dioxide, the gas would be led to flow into the microalgae culture unit, where carbon dioxide would be treated as a carbon source needed for microalgae's photosynthesis. Therefore, regardless of its source being either from biogas itself, or as a product produced from operation of electric power generation, carbon dioxide can be delivered to the microalgae culture unit, and if subject to light bath, the microalgae inside the microalgae culture unit will undergo photosynthesis in which carbon dioxide is affixed, thereby cutting down overall carbon dioxide discharge. At the same time, wastewater or waste substances and even microalgae produced by microalgae culture unit can all be used as the raw material for biogas production unit, and the microalgae produced therefrom can also be used as raw material of biomass fuel.

In other words, biogas produced by a biogas generation unit can all-in-once include hydrogen sulfide, methane, and carbon dioxide. Hydrogen sulfide that can easily cause damage to pipelines or generators would be removed by a biogas purification unit, carbon dioxide would be absorbed by the microalgae culture unit. As a result, the electric generator can effectively use methane to produce electricity, and the carbon dioxide generated subsequently by the electric generator can also be absorbed by microalgae culture unit, thereby enabling the entire generator with recycle-to-reuse capability and zero-carbon-emission functionality.

The abovementioned biogas electric generator of the present invention can optionally include: a heat recycling unit, which works to receive a thermal energy generated by the electric generator, and transfer the thermal energy to the biogas production unit, biogas purification unit, and microalgae culture unit at a temperature within a predetermined range, in order to facilitate the production of biogas and its purification, and the growth of microalgae.

In addition, the abovementioned biogas electric generator of the present invention can also optionally include: a biomass fuel production unit, wherein a portion of the microalgae of the microalgae culture unit is transferred to the biomass fuel production unit before its conversion into a biomass fuel and a waste substance through the biomass fuel generation unit. The waste substance is transferred to the biogas production unit for use as an anaerobic digestion raw material for producing biogas. It will be understood here that the biomass fuel described above can be, for example, biomass diesel fuel or biomass alcohol; the waste substance above can be algae residue or glycerol produced from the production process of biomass fuel.

In the abovementioned biogas electric generator of the present invention, the electric generator can further produce carbon dioxide, which is sent to the microalgae culture unit for use as carbon source for photosynthesis. In another aspect of the present invention, algae residue would be produced during the operation of the microalgae culture unit, wherein the residue is transferred to the biogas production unit for use as raw material in anaerobic digestion for producing biogas, for which the microalgae culture unit can be set up as open or close system. Moreover, the microalgae used by the microalgae culture unit is not particularly restricted, and is suitable for the current invention as it is insusceptible to the effect of highly concentrated methane gas, and can absorb carbon dioxide and engage in photosynthesis when it is exposed to light bath, so as to reduce the microalgae producing carbon dioxide in the biogas. At the same time in a preferred embodiment of the present invention, strains of Chlorella sp. are used as the microalgae described due to the more favorable carbon dioxide removal rate as effectuated by the mutation agent, the current invention is not particularly limited with this respect, commonly seen microalgae of wild species can also be used.

In the abovementioned biogas electric generator, the biogas production unit can be a single tank anaerobic digester, a double tank anaerobic digester, or a three-stage anaerobic digester. In the case of a three-stage anaerobic digester, the operating processes would involve hydrolysis, acidification, methanation, and others. The raw materials used in the biogas production unit can be waste substances discharged from the biomass fuel generation unit, wastewater or a portion of microalgae of the microalgae culture unit, or wastewater or waste substances obtained from agricultural sources. The residual solid substances resulting from the conversion of raw materials into biogas through anaerobic digestion can be used as organic fertilizer.

Furthermore, the biogas purification unit can use chemical means or physical means to remove or absorb hydrogen sulfide; and can be a biofiltering bed or a biotrickiling filter bed, wherein the filtering bed can be filled with peat, bark, vermiculite, oyster shells, zeolite, maifan stone, iron hydroxide, activated carbon, activated alumina, perlite, snake wood, and other materials offering microorganism immobilization advantage, for removing hydrogen sulfide; the process of removing hydrogen sulfide is known as desulfurization, it can work to not only avoid damage to pipeline or electric generator caused by hydrogen sulfide, but can also prevent hydrogen sulfide's impedance on the growth of microalgae.

Another object of the present invention is to provide a biogas electricity generation method using microalgae carbon capture, which leverages on the ability of microalgae to absorb carbon dioxide during photosynthesis to remove carbon dioxide in biogas, and to help streamline the uptake of carbon dioxide produced during the progress of electricity generation for microalgae. This form of usage demonstrates that the biomass fuel formed by microalgae can be an alternative to biogas for electricity generation, and can ultimately enhance electricity generation rate while also keep down greenhouse gases emitted during the electricity generation process.

In order to achieve the above object, another embodiment of the current invention is to provide a biogas electricity generation method using microalgae carbon capture, which comprises the following step: using a biogas production unit, which feeds a first biogas containing hydrogen sulfide, methane, and carbon dioxide to a biogas purification unit, where the biogas purification unit removes the hydrogen sulfide of the first biogas, and discharges a second biogas; guiding the second biogas to a microalgae culture unit, where the microalgae of the microalgae culture unit feeds on a carbon dioxide of the second biogas for carbon source for photosynthesis, and discharges a third biogas; and transferring the third biogas to an electricity generation unit, which uses the third biogas; and transferring the third biogas to an electricity generation unit, which uses the third biogas as a raw material to generate electrical power, which supplies the biogas electricity generator with power in order to operate the biogas electricity generator.

The biogas electricity generation method of the present invention mentioned above can optionally comprise the following additional steps: using a heat recycling unit to receive a thermal energy generated by the electricity generation unit, and transferring the thermal energy to the biogas production unit, the biogas purification unit, and the microalgae culture unit, so as to maintain temperatures of the biogas production unit, biogas purification unit, and microalgae culture unit within a predetermined range. The predetermined range can vary depending on different application subject, for which it is preferred to be the temperature ranges favorable for biogas production, biogas purification, or microalgae growth.

The abovementioned biogas electricity generation method of the current invention can further comprise the following steps: transferring a portion of the microalgae from the microalgae culture unit to the biomass fuel generation unit, which converts the portion of the microalgae into a biomass fuel and a waste substance, and guiding the waste substance to the biogas production unit for use as raw material in anaerobic digestion for producing biogas.

The biogas electricity generation method mentioned above can optionally comprise another step: transferring the waste gas of carbon dioxide generated by the electricity generation unit to the microalgae culture unit for use as a carbon source for photosynthesis.

The biogas electricity generation method of the current invention from the above can optionally comprise another step: transferring residues produced from the operation of the microalgae culture unit to the biogas production unit for use as raw material in anaerobic digestion for producing biogas.

In addition, for the biogas electric generator and its method of using of the current invention, it will be understood by persons having ordinary skills in the art that biomass fuel obtained from microalgae can be used as an alternative raw material in addition to the use of methane of biogas as raw material for electricity generation.

In view of the above, it will be apparent to skilled persons that the biogas electricity generator and its method of using disclosed herein involves the following. Biomass is obtained from microalgae culture unit (which in this case is microalgae), and the biomass can turn into biofuel through extraction or digestion (for which the products are biomass diesel fuel or biomass alcohol). Algae residue, glycerol, and other by-products produced can then enter biogas production unit to initiate anaerobic digestion in order to form biogas, carbon dioxide as resulting from biogas undergoing desulfurization can be used in growing microalgae, the remaining biogas (referring here to the remaining gas after carbon dioxide and hydrogen sulfide are removed) is used as raw material for generating electricity with electricity generator, and the carbon dioxide. The waste heat from the electricity generator can be recycled for other purposes including growing microalgae or increasing production capacity for biogas. Therefore, the entire system and its method can be attributed to a zero-carbon emission system, and its use method can be applied to environmental protection and energy related industries. It can be used to produce electricity or methane energy, biomass diesel fuel or biomass alcohol and various forms of energy, and the carbon dioxide discharged from energy production can be further recycled for use in growing microalgae, to ultimately achieve carbon balance, and decrease greenhouse gas production.

### BRIEF DESCRIPTION OF THE DRAWINGS

FIG. 1 is a schematic diagram illustrating a biogas electric generator according to a preferred embodiment of the current invention.

FIGS. 2(A) and 2(B) are graphs showing biogas concentration of preparative example 2 of the present invention, wherein, FIG. 2(A) is the case with presence of 0.08% (w/v) crude glycerol, and FIG. 2(B) is the case of presence of 0.01% (w/v) algae residue.

FIGS. 3(A) and 3(B) are experimental results with desulfurized biogas being shown in FIG. 3(A) and waste gas of electric generator being shown in FIG. 3(B).

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. Other various aspects also may be practiced or applied in the invention, and various modifications and variations can be made without departing from the spirit of the invention based on various concepts and applications.

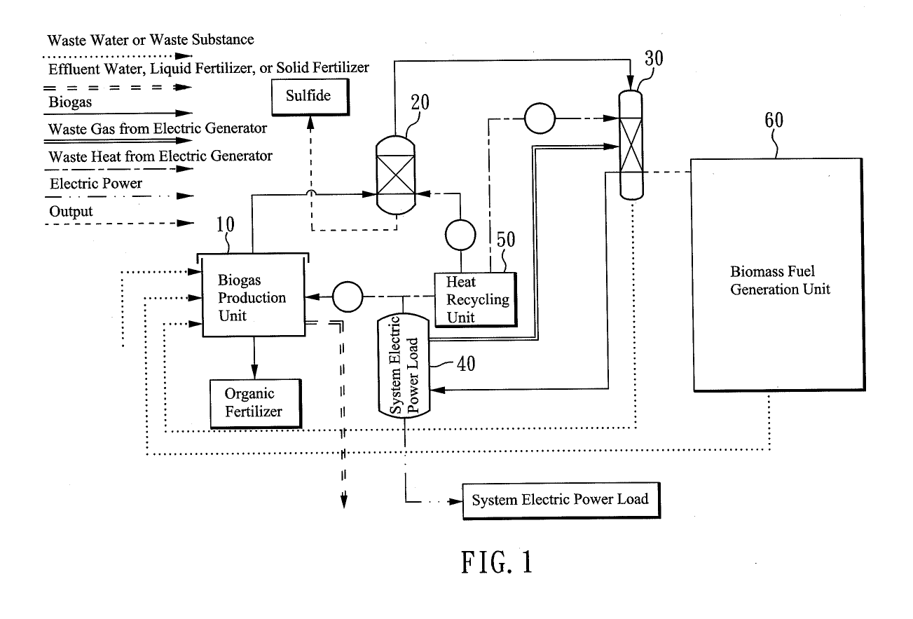

In reference to FIG. 1, a schematic diagram for the biogas electric generator of the present invention is presented. As can be seen in FIG. 1, the biogas electric generator of the present invention comprises: a biogas production unit 10, a biogas purification unit 20, a microalgae culture unit 30, an electricity generation unit 40, a heat recycling unit 50, and a biomass fuel generation unit 60.

The biogas production unit 10 can be a single tank anaerobic digester, a double tank anaerobic digester, or a three-stage anaerobic digester. In the case when the three-stage anaerobic digester is used, the processes carried out respectively would include hydrolysis, acidification and mathanation. The raw material required for the biogas production unit 10 can be waste substance discharged from biomass fuel generation unit 60, wastewater or waste substance from microalgae culture unit 30 or some microalgae, or wastewater or waste substance resulting from foreign agricultural sources. After the raw material is converted into biogas through anaerobic digestion, the residual solid substances can be used as organic fertilizers. The biogas produced at this time is a first biogas, which comprises hydrogen sulfide, methane and carbon dioxide.

Subsequently, the first biogas formed by the biogas production unit 10 is transferred to the biogas purification unit 20. The biogas purification unit 20 can utilize either a chemical means or physical means to remove or absorb hydrogen sulfide; the biogas purification unit can also be biofiltering bed or biotrickling bed, wherein the filtering beds above can be filled with activated carbon, peat, bark, vermiculite, oyster shells, zeolite, maifan stone, iron hydroxide, activated alumina, perlite, snake wood, and other materials offering microorganism (such as sulphur oxidation bacteria) immobilization advantages, so as to facilitate removal of hydrogen sulfide, and to not only avoid damage to pipeline or electric generator caused by hydrogen sulfide, but to also prevent hydrogen sulfide's impedance on the growth of microalgae. For this reason, after the biogas purification unit 20 is done with eliminating hydrogen sulfide of first biogas, the discharged gas would be known as second biogas, which mainly comprises methane and carbon dioxide.

Next, the second biogas will be guided to enter into the microalgae culture unit 30, where the microalgae culture unit 30 comprises a microalgae, for which there is in particular no restriction on the type of microalgae to be used, provided the chosen type is deflective against influences by highly concentrated methane gas, and can absorb carbon dioxide to carry out photosynthesis when subject to light bath, an example fitting this description would be the Chlorella sp. Accordingly, after the second biogas passes through the microalgae culture unit 30, the carbon dioxide concentration of the emitted gas can be lowered to its minimum. The emitted gas therein will be understood to be a third biogas. After the microalgae culture unit 30 continues to operate for a period of time, the residue produced during the on-time of the process or a portion of the microalgae can be transferred to the biogas production unit 10, for adding to the raw material for producing biogas.

Subsequently, the third biogas is transferred to the electricity generation unit 50 for use as raw material for generating an electrical energy, a thermal energy and a waste gas comprising carbon dioxide. In the present case, the electrical energy is used for supplying the entire biogas electricity generator with the electrical power for running regular operation, and the waste gas produced in the electricity generation process is also transferred to the microalgae culture unit 30, so as to reduce the carbon dioxide concentration in the waste gas to a minimum level. In another perspective of the current invention, the thermal energy produced is recycled by the heat recycling unit 50, so as to utilize the thermal energy on maintaining or raising the temperatures of biogas production unit, the biogas purification unit, and the microalgae culture unit.

The microalgae produced by the microalgae culture unit 30 can be transferred to the biogas fuel generation unit 60, and gets converted into a biomass fuel (such as biomass diesel fuel or biomass alcohol) and a waste substance (such as algae residue or glycerol), the waste substance can also be transferred to the biogas production unit 10.

It should now therefore be understood from the above description that the carbon source used in the biogas production unit 10 can come from various types of waste substances, which generates biogas from these materials through anaerobic digestion, and the biogas then enters into the biogas purification unit 20 for removal of hydrogen sulfide, in order to decrease the corrosion of hydrogen sulfide on the electricity generator. The desulfurized biogas then enters into the microalgae culture unit 30, uses microalgae to absorb carbon dioxide to emulate the characteristics for the carbon source for growth purposes, in order to achieve the object of reducing carbon. The growth microalgae can further work to produce biomass diesel fuel, biomass alcohol or enters into anaerobic biogas production unit 10, the algae residue or glycerol from production of biomass diesel fuel or biomass alcohol can further be placed into the biogas production unit 10 to be converted into biogas. The purified biogas can undergo the treatment of electricity generation unit 40 to proceed with electricity generation. The carbon dioxide and waste heat produced from the electricity generation unit 40 can be recycled to support microalgae growth or increase biogas production capacity and other purposes.

### Experimental Example 1

Referring now to A Mutant Strain of Microalga Chlorella sp. for the Carbon Dioxide Capture from Biogas by Kao C—Y et al., Biomass and Bioenergy (2012) 36: 132-140, preparation and growing of Chlorella sp., wherein the culture medium is a modified f/2 medium, which is arranged by man-made seawater, and comprises 29.23 g/L NaCl, 1.105 g/L KCl, 11.09 g/L MgSO₄.7H₂O, 1.21 g/L Tris-base, 1.83 g/L CaCl₂.2H₂O and 0.25 g/L NaHCO₃ and at the same time comprises 0.3% (v/v) macro elemental solution and 0.3% trace elemental solution, wherein the trace elemental solution has 4.36 g/L Na₂.EDTA, 3.16 g/L FeCl₃.6H₂O, 180 mg/L MnCl₂.4H₂O, 10 mg/L CoCl₂.6H₂O, 10 mg/L CuSO₄.5H₂O, 23 mg/L ZnSO₄.7H₂O, 6 mg/L Na₂MoO₄.2H₂O, 100 mg/L vitamin B1, 0.5 mg/L vitamin B₁₂ and 0.5 mg/L biotin. The initial pH value of the culture medium sits between 7.4 and 7.6.

Acrylic column is used as a Chlorella sp. growing column, whose length is 2.5 m, diameter is 20 cm, and working volume is 40 L. Swine wastewater is used to proceed with anaerobic digestion to produce biogas and the methane, carbon dioxide and nitrogen in methane, with each having a volume of about 70±5%, 20±2% and 8±3%. After the biogas is desulfurized by chemical adsorption, the concentration of hydrogen sulfide is lowered to be below 100 ppm. During the growing phase, biogas and atmospheric gas are introduced to enter into the column for 30 minutes respectively on each day. Gas flow rate is set to be 0.1 vvm and kept constant for 8 hours, while also concentration sampling of carbon dioxide and methane is taken on the inflow and outflow of biogas. More particularly, removal of carbon dioxide (%) is calculated by the following formula:

((Percentage of Co₂ Intake−Percentage of CO₂ output)/Percentage of CO₂ Intake)×100%

As could be demonstrated by the experimental results put forth below in Table 1, desulfurized biogas that goes through microalgae culture unit can effectively absorb 80% carbon dioxide in 10 minutes, and moves the removal rate to 51% under 20 minutes of continuous aeration; furthermore, content of methane can be brought up from 71% to 87% during the 10 minutes of aeration. This is indicative of the increase of methane, and it also means improved electricity generation efficiency.

TABLE 1
Clear Day (approximately 1500 μmol⁻²s⁻¹)

| | Biogas Aeration Period | | |
|---|---|---|---|
| | 10 Minutes | 20 Minutes | 30 Minutes |
| Percentage of | 20 ± 1.0 | 20.0 ± 0.5 | 20.0 ± 1.0 |
| CO₂ Intake (%) | | | |
| Percentage of | 4.1 ± 0.8 | 9.8 ± 0.8 | 14.2 ± 1.0 |
| CO₂ Output | | | |
| (%) | | | |
| Percentage of | 80 | 51 | 29 |
| CO₂ Removal | | | |
| (%) | | | |
| Percentage of | 71.1 ± 2.5 | 69.6 ± 2.9 | 69.0 ± 2.1 |
| CH₄ Intake (%) | | | |
| Percentage of | 87.4 ± 2.3 | 80.3 ± 4.0 | 75.3 ± 1.8 |
| CH₄ Output | | | |
| (%) | | | |

### Experimental Example 2

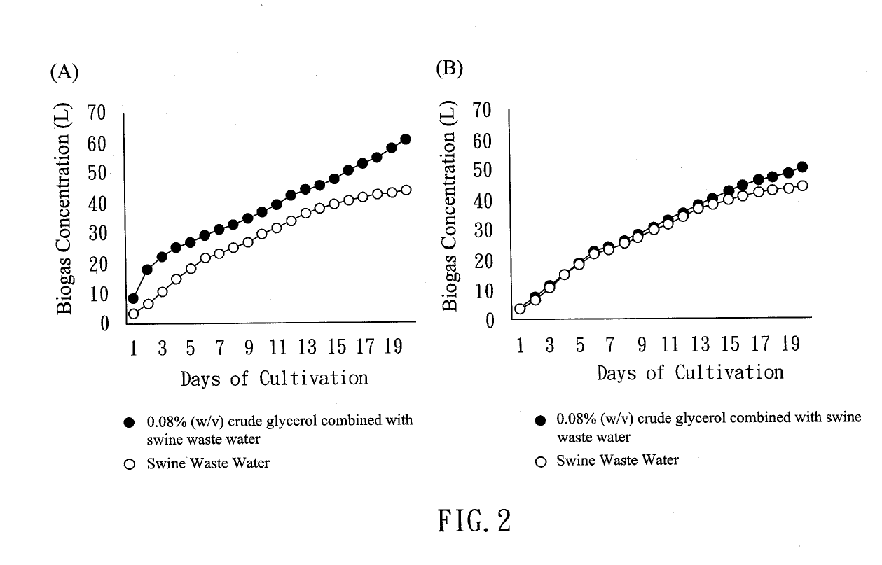

Referring now to Taiwan Patent No. M410052, whose title is translate into “Wastewater Treatment Device for Effectively Producing Methane.” The experimental example here involves adding respectively grown microalgae or residue or crude glycerol, both of which extracted from biomass diesel fuel, into wastewater treatment device, using batch-based culturing to produce biogas, and compares biogas production results as coming from regular wastewater and from presence of algae residue or crude glycerol. More particularly, FIG. 2(A) shows the experimental result from comparing presence of 0.08% (w/v) crude glycerol, FIG. 2(B) shows the experimental result from comparing present of 0.01% (w/v) algae residue. As shown by FIG. 2, in the situation where hydropower is stayed for 8 days, and for 0.8 g crude glycerol and 0.1 algae residue added per liter, the biogas production capacity can be raised respectively to 39% and 14%, for which the conversion result shows for each gram of added crude glycerol, 700 mL of biogas can be produced, and each gram of algae residue can produce 125 mL of biogas.

In summary of the above description, the present invention discloses a zero-carbon-emission oriented biogas electric generator and its method, wherein the generator includes the anaerobic biogas production unit, biogas purification unit, methane-tolerant and hydrogen sulfide-tolerant microalgae culture unit, electricity generation unit and waste heat recycling unit. The function of the anaerobic biogas production unit is to process the various kinds of waste substances, and converts the carbon source into methane, turns left-overs into organic fertilizers. Biogas purification unit is used to remove hydrogen sulfide in biogas, and ultimately lower the risk of corrosion on the integrity of the electricity generator. The microalgae culture unit works to use biogas or carbon dioxide generated by electricity generation unit to grow microalgae, in order to reduce carbon. The grown microalgae would be further used to produce biomass diesel fuel, biomass alcohol or can enter into anaerobic biogas production unit. The purified biogas can turn to be used to generate electricity through the working of electricity generation unit, and the waste heat produced from the electricity generator can be recycled to provide for growing of microalgae or improving biogas production. It will be understood here then, that the current invention can recycle and reuse various kinds of waste heat, waste water, waste gas or waste substances, for achieving the zero-carbon emission based environmental protection purposes.

### Experimental Example 3

In another embodiment, an acrylic column having a length of 3 m, a diameter of 16 cm, a working volume of 50 L is used as a Chlorella sp. growing column. Sources of gas introduced hereinto include: 1. Biogas produced from swine waste water, the carbon dioxide content in the biogas is 20±5%, after the biogas is desulfurized by way of chemisorption, or biofiltration concentration of hydrogen sulfide is lowered to be below 100 ppm, 2. Waste gas produced from electric generator, the carbon dioxide content in the waste gas is 15±0.6%. Biogas and air are each given 30 minutes to be passed into microalgae group column, of which five columns are included, wherein biomass concentration are 0.5 g/L, 1 g/L, 1.5 g/L, 2 g/L and 2.5 g/L, respectively. When gas flow rate is 0.05 vvm, 8 cycles are continually kept watched, and carbon dioxide concentration in the gas outflow and inflow are sampled.

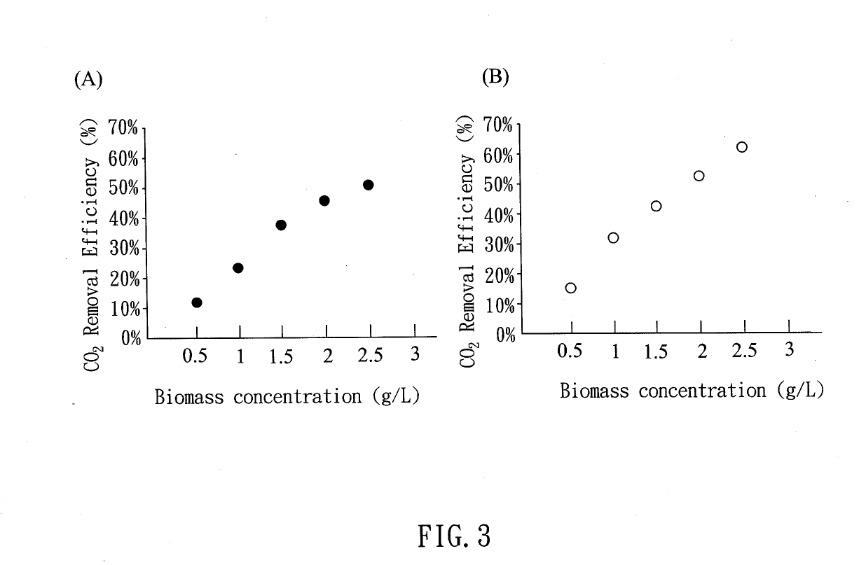

As indicated by the experimental results in FIG. 3, desulfurized biogas in FIG. 3(a) is passed into 0.5 g/L, 1 g/L, 1.5 g/L, 2 g/L and 2.5 g/L biomass concentration microalgae culture system, carbon dioxide removal efficiency is 13%, 26%, 38%, 46% and 53%, respectively; waste gas of electric generator in FIG. 3(b) is passed into 0.5 g/L, 1 g/L, 1.5 g/L, 2 g/L and 2.5 g/L biomass concentration microalgae culture system, carbon dioxide removal efficiency is 16%, 32%, 43%, 53% and 61%, respectively. The result is an indication that Chlorella sp. can effectively remove carbon dioxide in the desulfurized biogas and waste gas of electric generator. Higher Chlorella sp. concentration in the microalgae culture system can effectively remove more carbon dioxide.

The abovementioned examples described herein are for illustrative purposes only, it is therefore intended that the scope of coverage provided for the current invention be determined only by the language of the claims in the current application, and not limited by the above examples.

What is claimed is:

1. A biogas electric generator using microalgae carbon capture, comprising:
(A) a biogas production unit, which provides a first biogas, wherein, the first biogas comprises hydrogen sulfide, methane and carbon dioxide;
a biogas purification unit, which receives the first biogas and removes the hydrogen sulfide of the first biogas, and discharges a second biogas;
a microalgae culture unit, which houses a type of microalgae and receives the second biogas, wherein, the microalgae uses the carbon dioxide of the second biogas as carbon source to carry out photosynthesis before the microalgae culture unit discharges a third biogas; and
an electric generation unit, which receives the third biogas for use as a fuel to generate electric energy, wherein, the electric energy powers the biogas electric generator.

2. The biogas electric generator using microalgae carbon capture according to claim 1, further comprising a heat recycling unit, which works to receive a thermal energy generated by the electric generator, and transfer the thermal energy to the biogas production unit, biogas purification unit, and microalgae culture unit.

3. The biogas electric generator using microalgae carbon capture according to claim 2, further comprising a biomass fuel generation unit, wherein, the microalgae of the microalgae culture unit is transferred to the biomass fuel generation unit, and is then converted into a biomass fuel and a waste substance, the waste substance is transferred to the biogas production unit.

4. The biogas electric generator using microalgae carbon capture according to claim 2, wherein, the electricity generation unit further produces a carbon dioxide containing waste gas, which is transferred to the microalgae culture unit.

5. The biogas electric generator using microalgae carbon capture according to claim 2, wherein, the microalgae culture unit produces algae residue during the operation of the microalgae culture unit, the residue is transferred to the biogas production unit.

6. A biogas electricity generation method using microalgae carbon capture, comprising the steps of:
using a biogas production unit, which feeds a first biogas containing hydrogen sulfide, methane, and carbon dioxide to a biogas purification unit, where the biogas purification unit removes the hydrogen sulfide of the first biogas, and discharges a second biogas;
guiding the second biogas to a microalgae culture unit, where the microalgae of the microalgae culture unit feeds on a carbon dioxide of the second biogas for carbon source for photosynthesis, and discharges a third biogas; and
transferring the third biogas to an electricity generation unit, which uses the third biogas as a raw material to generate electrical power, which supplies the biogas electricity generator with power in order to operate the biogas electricity generator.

7. The biogas electricity generation method using microalgae carbon capture according to claim 6, further comprising the step of using a heat recycling unit to receive a thermal energy generated by the electricity generation unit, and transferring the thermal energy to the biogas production unit, the biogas purification unit, and the microalgae culture unit

8. The biogas electricity generation method using microalgae carbon capture according to claim 7, further comprising the step of transferring a portion of the microalgae from the microalgae culture unit to the biomass fuel generation unit, which converts the portion of the microalgae into a biomass fuel and a waste substance, and guiding the waste substance to the biogas production unit.

9. The biogas electricity generation method using microalgae carbon capture according to claim 7, further comprising the step of transferring the waste gas of carbon dioxide generated by the electricity generation unit to the microalgae culture unit

10. The biogas electricity generation method using microalgae carbon capture according to claim 7, further comprising the step of transferring residues produced from the operation of the microalgae culture unit to the biogas production unit
